(19) United States
(12) Patent Application Publication
(10) Pub. No.: US 2002/0169497 A1
(43) Pub. Date: Nov. 14, 2002

(54) Endovascular stent system and method of providing aneurysm embolization

(76) Inventors: Petra Wholey, San Antonio, TX (US); Michael Wholey, San Antonio, TX (US); Mark Wholey, Oakmont, PA (US)

Correspondence Address: Timothy D. Broms, Pietragallo, Bosick & Gordon, One Oxford Centre, 38th Floor, 301 Grant Street, Pittsburgh, PA, 15219, US

(21) Appl. No.: 10/152,928

(22) Filed: May 22, 2002

Related U.S. Application Data
(63) Continuation-in-part of application No. 09/753,119, filed on Jan. 2, 2001.
(60) Provisional application No. 60/292,557, filed on May 22, 2001.

(51) Int. Cl. A61F 2/06

(52) U.S. Cl. 623/1.13; 623/1.16; 606/200

(57) ABSTRACT

The present invention relates to an improved endovascular stent and stent graft, which may be combined to form a stent system. The stent system may be used to treat aneurysms, such as abdominal aortic aneurysms. The stent system includes at least one port located in the sidewalls of the stent and/or stent graft to allow for the delivery of embolic devices to locations outside of the stent system. Methods of utilizing the stent system for embolizing an aneurysm are also provided.

### CROSS-REFERENCE TO RELATED APPLICATIONS

This application is a continuation-in-part of U.S. application Ser. No. 09/753,119 filed Jan. 2, 2001. This application also claims the benefit of U.S. Provisional Application Serial No. 60/292,557 filed May 22, 2001.

### FIELD OF THE INVENTION

The present invention relates to endovascular stents and endovascular stent grafts, and more particularly relates to endovascular stents and endovascular stent grafts with ports extending through the sidewalls of the grafts to allow for the placement of catheters and other delivery devices which allow for the injection of embolic agents and other materials to locations outside of the grafts. Methods of utilizing the endovascular stent and endovascular stent graft to treat aneurysms are also provided.

### BACKGROUND INFORMATION

Various types of endovascular stents and endovascular stent grafts are used in the field of endovascular surgery. When an individual develops an aneurysm, there is a high risk of rupture. Treatment options include surgery or endovascular stent and/or endovascular stent graft placement. One of the limitations of stent and stent graft placement is the risk of continued enlargement of the aneurysm. Enlargement of the aneurysm occurs from a leakage, sometimes referred to as an endoleak. The risk for leakage can occur in up to 20-30% of all stent and stent graft placements. If the endoleak goes unchecked, there is the potential risk for rupture of the aneurysm. Endoleaks can occur from the proximal or distal ends of stents and stent grafts, where a poor seal can occur resulting in blood passing around the stent and/or stent graft into the aneurysm. Leaks can occur through defects of the stent material. Leaks can also occur from collateral vessels that begin above and below the aneurysm, sending vessels back into the aneurysm. For example, sometimes the lumbar artery will reform from the internal iliac artery. These lumbar branches may then flow retrograde or backwards into the aneurysm.

If a modular stent system is used, there is also the potential problem of migration of the modular components of the stent system. Migration can occur with segments moving apart over time due to repeated stress forces and dynamics. This migration of the modular components can also lead to endoleaks.

A need exists for an endovascular stent and/or endovascular stent graft that substantially reduces the possibility of endoleaks and reduces the risk for rupture of an aneurysm. A need also exists for a modular stent system that prevents the possible migration of the modular components of the stent system.

The present invention has been developed in view of the foregoing, and to address other deficiencies of the prior art.

### SUMMARY OF THE INVENTION

An aspect of the present invention is to provide an endovascular stent including a lumen having a sidewall, and at least one port extending through the sidewall of the lumen structured and arranged to allow for the delivery of an embolic device outside the stent.

Another aspect of the present invention is to provide an endovascular stent including a lumen having a sidewall, and means to allow for the delivery of an embolic device outside the stent and into a space occupied by an aneurysm.

A further aspect of the present invention is to provide an endovascular stent graft including a lumen having a sidewall and having a distal section, and proximate ipsilateral and contralateral sections structured to be disposed within iliac arteries, and at least one port extending through the sidewall of the lumen structured and arranged to allow for delivery of an embolic device outside the stent graft.

Another aspect of the present invention is to provide an endovascular stent system including an endovascular stent having a lumen including a sidewall and at least one port extending through the sidewall of the lumen structured and arranged to allow for delivery of an embolic device outside the stent, and an endovascular stent graft having a lumen including a sidewall and having a distal section and proximate ipsilateral and contralateral sections structured to be disposed within iliac arteries and at least one port extending through the sidewall of the lumen structured and arranged to allow for delivery of an embolic device outside the stent graft.

A further aspect of the present invention is to provide a method of using an endovascular stent for embolizing an aneurysm. The method includes the steps of providing an endovascular stent having a lumen wherein the lumen includes a perimeter and sidewalls extending around substantially the entire perimeter and wherein the sidewalls include at least one port extending through the sidewalls, positioning the endovascular stent within an artery adjacent to an aneurysm, guiding a delivery device into the endovascular stent, inserting the delivery device into the port so that at least a portion of the delivery device extends through the sidewall of the lumen, and delivering material through the delivery device to a location outside the stent.

Another aspect of the present invention is to provide a method of using an endovascular stent graft for embolizing an aneurysm. The method includes the steps of providing an endovascular stent graft having a distal section, proximate ipsilateral and contralateral sections, and a lumen wherein the lumen includes a perimeter and sidewalls extending around substantially the entire perimeter and wherein the sidewalls include at least one port extending through the sidewalls, positioning the endovascular stent graft within an artery adjacent to an aneurysm, guiding a delivery device into the endovascular stent graft, inserting the delivery device into the port so that at least a portion of the delivery device extends through the sidewall of the lumen, and delivering material through the delivery device to a location outside the stent graft.

A further aspect of the present invention is to provide a method of using an endovascular stent system for embolizing an aneurysm. The method includes the steps of providing an endovascular stent system having an endovascular stent having a lumen including a sidewall with at least one port extending therethrough, and an endovascular stent graft having a lumen including a sidewall and having a distal section, and proximate ipsilateral and contralateral sections, wherein the sidewall includes at least one port extending therethrough, positioning the endovascular stent system within an artery adjacent to an aneurysm, guiding a delivery device into the endovascular stent system, inserting the delivery device into the port of the stent or the port of the stent graft so that at least a portion of the delivery device extends through the sidewall of the stent or the sidewall of the stent graft, and delivering an embolic device through the delivery device to a location outside the stent system.

These and other aspects of the present invention will be more apparent from the following description.

### BRIEF DESCRIPTION OF THE DRAWINGS

FIG. 1 is a partially schematic illustration of an endovascular stent in accordance with an embodiment of the present invention.

FIG. 2 is a partially schematic illustration of an endovascular stent graft in accordance with an embodiment of the present invention.

FIG. 3 is a partially schematic illustration of a stent system in accordance with an embodiment of the present invention.

FIG. 4 is a partially schematic illustration of a stent system in accordance with another embodiment of the present invention.

FIG. 5 is a partially schematic illustration of an endovascular stent graft with a delivery device extending through a port in accordance with an embodiment of the present invention.

FIG. 6 is a partially schematic illustration of the endovascular stent graft of FIG. 5 with the delivery device delivering an embolic agent into a space occupied by an aneurysm.

FIG. 7 is a partially schematic illustration of an endovascular stent system with a delivery device delivering embolic agents into a space occupied by an aneurysm of an iliac artery in accordance with an embodiment of the present invention.

FIG. 8 is a side sectional view of a portion of an endovascular stent sidewall with a one-way valve assembly in accordance with an embodiment of the present invention.

FIG. 9 is a cross-sectional view of the one-way valve assembly of FIG. 8.

FIG. 10 is a side sectional view of a portion of an endovascular stent sidewall with a one-way valve assembly in accordance with another embodiment of the present invention.

FIG. 11 is a cross-sectional view of the one-way valve assembly of FIG. 10.

FIG. 12 is a side sectional view of a portion of an endovascular stent sidewall with a one-way valve assembly in accordance with another embodiment of the present invention.

FIG. 13 is a cross-sectional view of the one-way valve assembly of FIG. 12.

FIG. 14 is a side sectional view of a portion of an endovascular stent sidewall with a one-way valve assembly in accordance with another embodiment of the present invention.

FIG. 15 is a cross-sectional view of the one-way valve assembly of FIG. 14.

FIG. 16 is a side sectional view of a portion of an endovascular stent sidewall with a guide wire piercing a port in accordance with an embodiment of the present invention.

FIG. 17 is a side sectional view of a portion of an endovascular stent sidewall with a delivery device extending through a port in accordance with another embodiment of the present invention.

FIG. 18 is a side sectional view of a portion of an endovascular stent sidewall with a guide wire piercing the sidewall in accordance with another embodiment of the present invention.

FIG. 19 is a partially schematic illustration of a sidewall of an endovascular stent including a one-way valve assembly in accordance with an embodiment of the present invention.

FIG. 20 is a partially schematic illustration of an endovascular stent in accordance with another embodiment of the present invention.

FIG. 21 is a partially schematic illustration of an endovascular stent graft in accordance with another embodiment of the present invention.

FIG. 22 is a partially schematic illustration of a stent system in accordance with another embodiment of the present invention.

FIG. 23 is a partially schematic illustration of a stent system with a delivery device delivering an embolic agent into a space occupied by an aneurysm in accordance with another embodiment of the present invention.

FIG. 24 is a partially schematic illustration of the stent system of FIG. 23 with the embolic agent completely filling the space occupied by an aneurysm.

FIG. 25 is a partially schematic illustration of an endovascular stent in accordance with another embodiment of the present invention.

FIG. 26 is a partially schematic illustration of an endovascular stent graft in accordance with another embodiment of the present invention.

FIG. 27 is a partially schematic illustration of a stent system in accordance with another embodiment of the present invention.

FIG. 28 is a partially schematic illustration of an endovascular stent graft with a cavity between the sidewalls of the stent graft and an outer covering being filled with an embolic agent in accordance with an embodiment of the present invention.

FIG. 29 is a partially schematic illustration of an endovascular stent graft with a cavity filled with an embolic agent and the length, width, and diameter of the outer covering has increased.

FIG. 30 is a partially schematic illustration of stent system with a cavity filled with an embolic agent and the length, width, and diameter of the outer covering has increased.

### DETAILED DESCRIPTION OF THE INVENTION

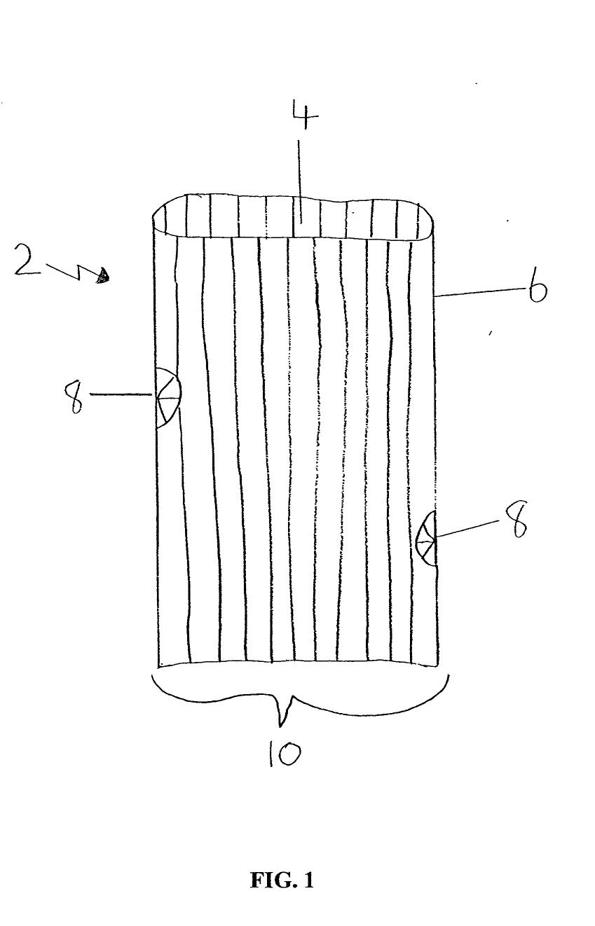

FIG. 1 illustrates an endovascular stent 2 in accordance with an embodiment of the present invention. The endovascular stent 2 includes a single lumen 4, sidewalls 6 which substantially extend around the perimeter of the lumen 4, and ports 8 for allowing an embolic device or embolic agent, such as a medical grade glue, to be delivered outside of the stent 2. As used herein, the term “endovascular stent” refers to a hollow, tubular, open-ended device used to maintain vascular patency in humans and animals. The endovascular stent 2 may be of a fixed diameter or of a variable diameter, both of which are known in the art, and the sidewalls 6 of the endovascular stent 2 may be of any standard construction known in the art. As used herein, the term “embolic device” refers to any suitable device or material that would be used in the field of cardiology for treating aneurysms, such as but not limited to embolic agents, suction for aspirating thromboembolic material, or diagnostic instruments. As used herein, the term “embolic agent” refers to, but is not limited to glue, saline, gel, silicone rubbers, urethanes and other organic elastomers, aqueous acyromide monomers, polymerizable protein solutions, cross-linked polyvinyl alcohol foam, polyurethane foam, polyethylene foam, silicone foam, fluorinated polyolefin foam, and/or an ethylene-vinyl-alcohol copolymer sold under the designation ONYX by MICRO THERAPEUTICS, INC.

In FIG. 1 the means to allow for the delivery of an embolic device to a location outside the stent 2 is one or more ports 8, but it is to be understood that any means such as valves, channels, or passages suitable for allowing an embolic device to be delivered through the sidewall of the stent to a location outside the stent is within the scope of the present invention. The endovascular stent 2 also includes a caudal section 10. The caudal section 10 may be structured so that it can be attached to a stent graft. The caudal section 10 of the endovascular stent 2 may be attached to a stent graft in a permanent fashion, or in a modular fashion where the stent and stent graft may be separated and re-attached.

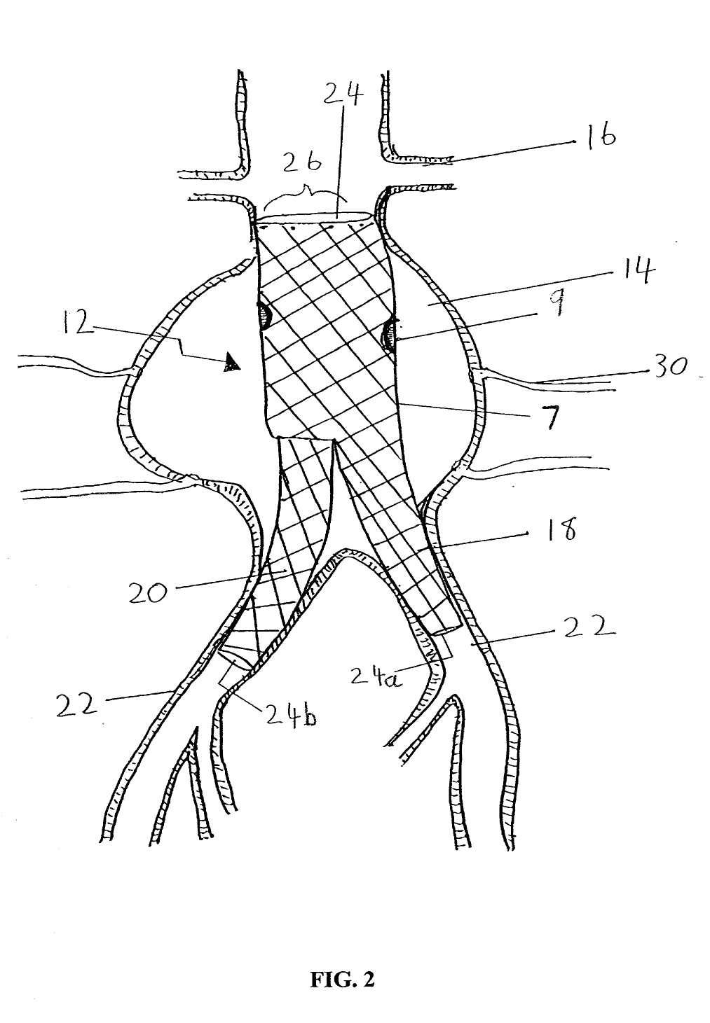

FIG. 2 illustrates an endovascular stent graft 12 in accordance with another embodiment of the present invention. The stent graft 12 includes a distal section 26 for allowing a stent to be attached, a second lumen 24 having a perimeter and sidewalls 7 extending around substantially the entire perimeter of the second lumen 24, an ipsilateral section 18 and a contralateral section 20, and ports 9 extending through the sidewalls of the second lumen for allowing an embolic device to be delivered outside of the stent graft 12. The lumen 24 also has a narrowed section 24a extending through the ipsilateral section 18 and a narrowed section 24b extending through the contralateral section 20. As used herein, the term “endovascular stent graft” refers to a hollow, tubular medical device designed to be placed within a blood vessel having a weakened vessel wall so as to repair the damaged section of the vessel by providing a new passageway through which blood or other matter may flow. The sidewalls 7 of the endovascular stent graft 12 may be of any standard construction known in the art. As shown in FIG. 2, the endovascular stent graft 12 may be placed into a position of an abdominal aortic aneurysm 14 at the level of the renal artery origins 16. The ipsilateral section 18 and the contralateral section 20 are structured to be seated within the iliac arteries 22.

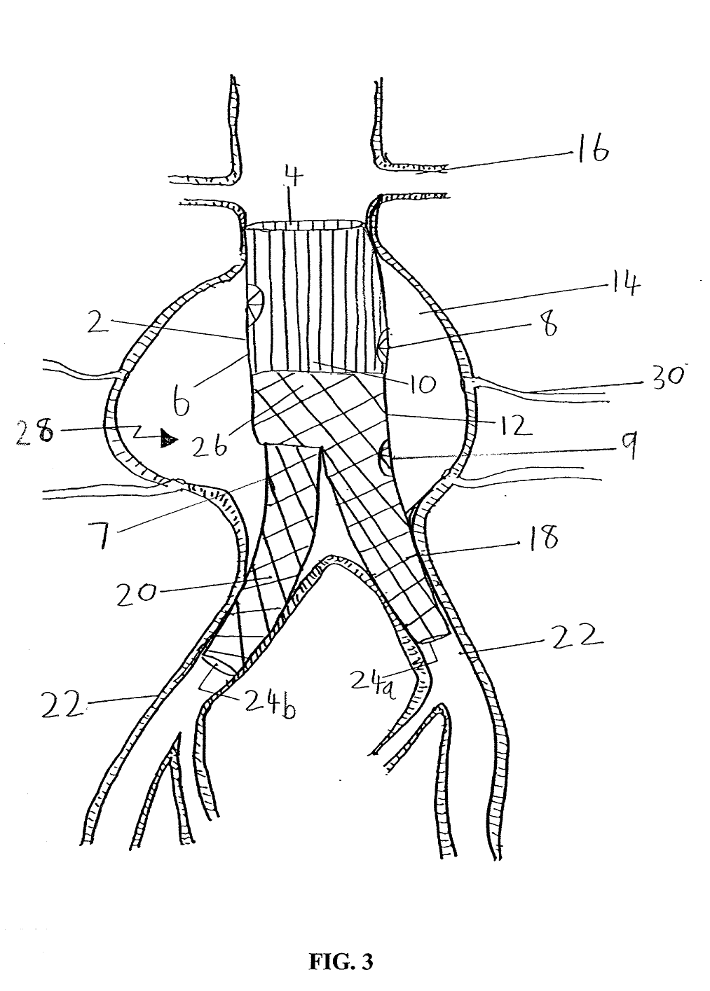

FIG. 3 illustrates an endovascular stent system 28, which includes the endovascular stent 2 shown in FIG. 1 and at least one endovascular stent graft 12 as shown in FIG. 2. As used herein, the term “endovascular stent system” refers to a system comprising an endovascular stent and at least one endovascular stent graft. The distal section 26 of the endovascular stent graft 12 may be attached to the caudal section 10 of the endovascular stent 2 permanently, or temporarily in a modular fashion. As shown in FIG. 3, the sidewall 6 of the first lumen 4 of the endovascular stent 2 and/or the sidewall 7 of the second lumen 24 of the endovascular stent graft 12 are structured to include at least one port 8 or 9 extending through the sidewalls 6 or 7 to allow for an embolic device to be delivered outside of the stent system 28.

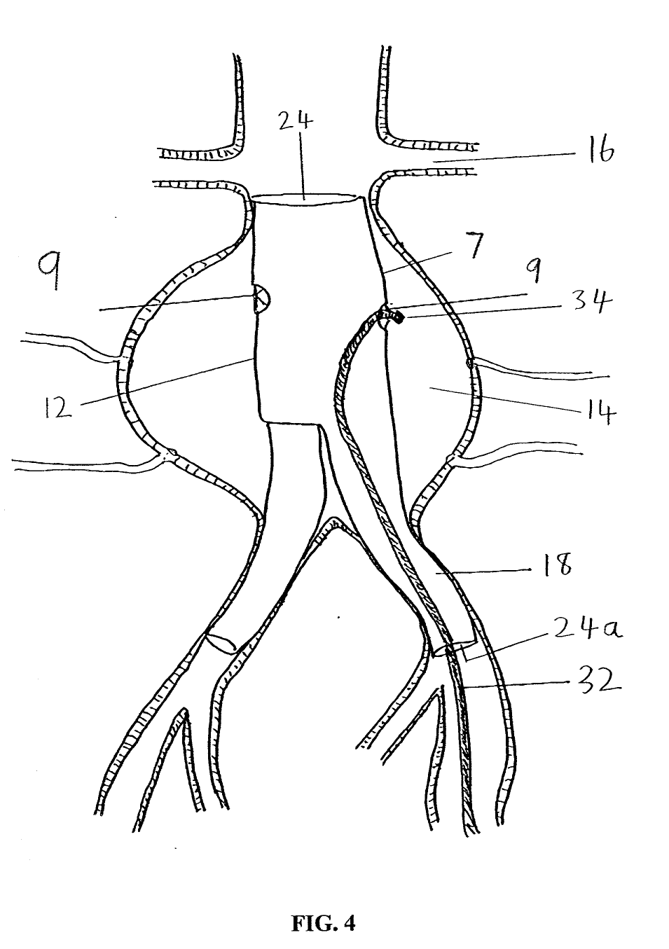

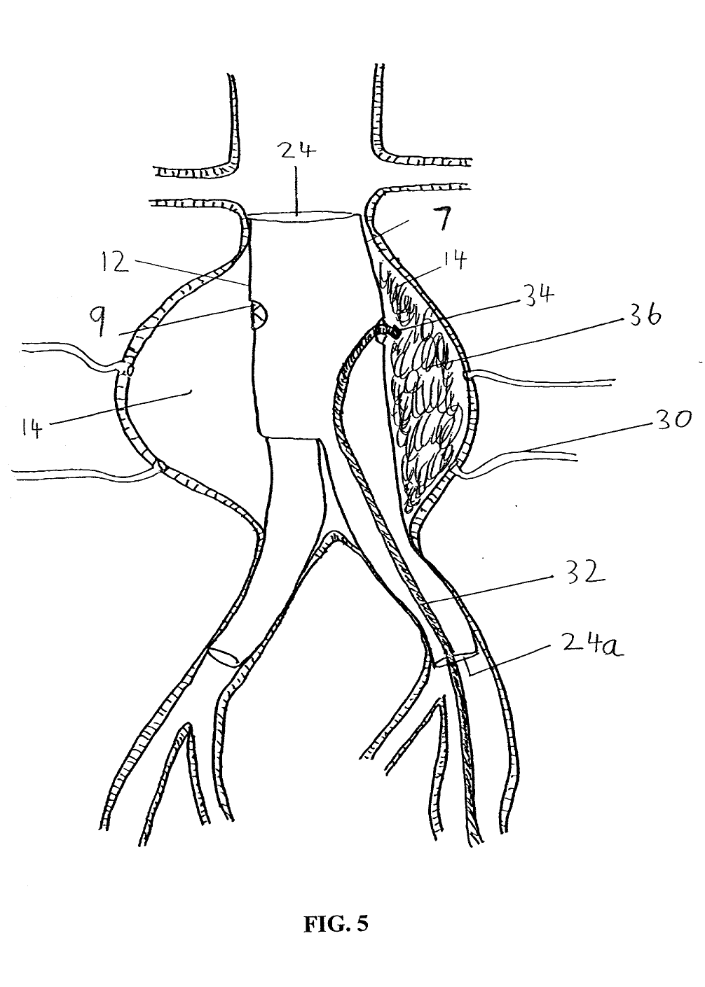

FIGS. 4 and 5 show an endovascular stent graft 12 of the present invention being used to treat an abdominal aortic aneurysm 14. FIG. 4 shows the endovascular stent graft 12 placed into a position of an abdominal aortic aneurysm 14 at the level of the renal artery origins 16. FIG. 4 also shows a delivery device 32 inserted into the ipsilateral section 18 of the endovascular stent graft 12 through the narrowed section 24a of the second lumen 24. As used herein, the term “delivery device” may be defined as, but is not limited to, a catheter, a medical grade tube, or a guide wire. As shown in FIG. 4, the delivery device 32 may be situated so that the tip 34 of the delivery device 32 extends through the port 9 and the sidewall 7. As shown in FIG. 4, at least a portion of the delivery device 32 extends into a space occupied by an aneurysm 14. The delivery device 32 may optionally lock into the port 9 to keep the delivery device 32 in place.

FIG. 5 shows an embolic agent 36, such as a medical grade glue, being delivered through the delivery device 32 and into the space occupied by an aneurysm 14. As shown in FIG. 5, the embolic agent 36 is delivered until the space occupying the aneurysm 14 is substantially filled with the embolic agent 36. By filling the space occupied by the aneurysm 14 with an embolic agent 36, thrombosis will be promoted and endoleaks will be prevented from occurring, especially endoleaks due to collateral vessels that may begin immediately above and below the aneurysm. The embolic agent 36 may also help to hold the modular components of a stent system together and in place, when such a stent system is used with the present invention.

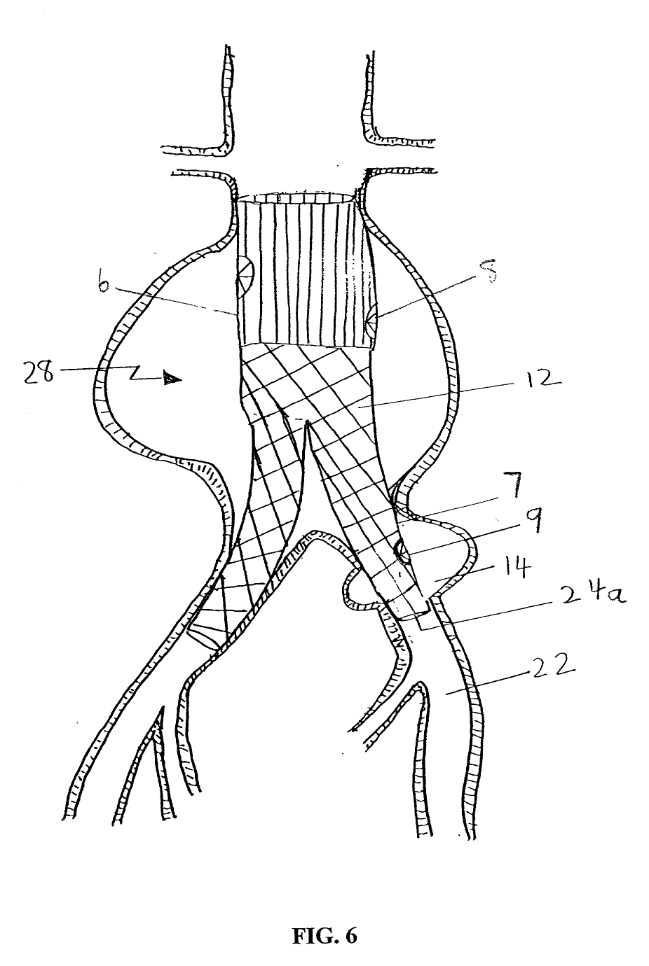

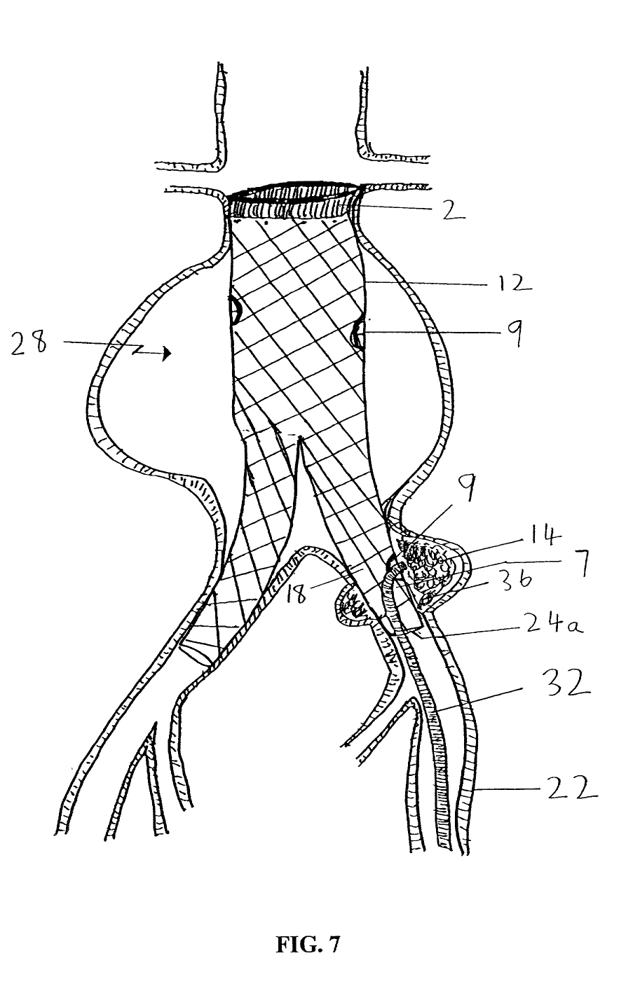

FIGS. 6 and 7 show a stent system 28 of the present invention being used to treat an aneurysm 14 of an iliac artery 22. As shown in FIG. 6, a port 9 is located within the sidewall 7 of the second lumen 24 of the endovascular stent graft 12 of the stent system 28 to allow for an embolic agent to be delivered through the port 9 and into a space occupied by the aneurysm 14 of an iliac artery 22.

As shown in FIG. 7, a delivery device 32 is inserted into the ipsilateral section 18 of the endovascular stent graft 12 through the narrowed section 24a of the second lumen 24 so that a portion of the delivery device 32 extends through the port 9 and into the space occupied by the aneurysm 14. An embolic agent 36 is injected through the delivery device 32 and substantially fills the space occupied by the aneurysm 14 of an iliac artery 22.

The ports of the present invention may be any suitable structure that allows embolic devices to be delivered outside of an endovascular stent 2, an endovascular stent graft 12, or a stent system 28.

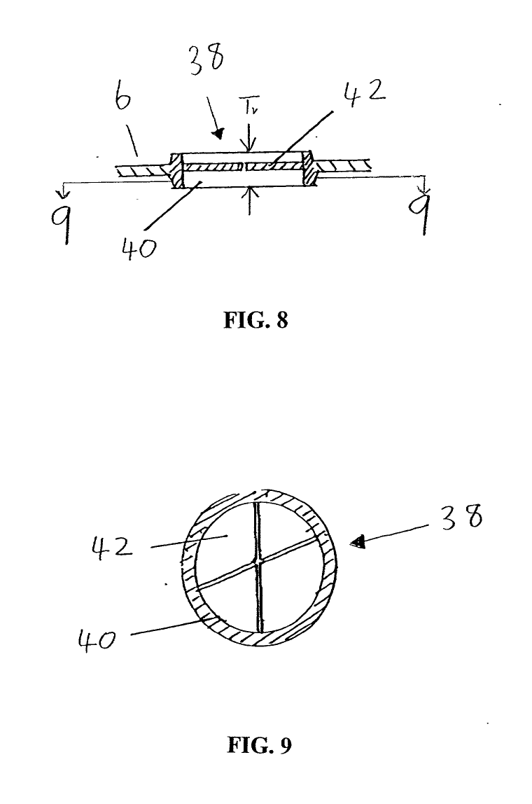

In a particular embodiment of the invention, the ports 8 or 9 may be one-way valve assemblies. FIG. 8 shows a side sectional view of a portion of a sidewall 6 of a stent 2 containing a one-way valve assembly 38. The one-way valve assembly 38 includes a valve housing 40 and valve flaps 42. Preferably, the valve flaps 42 are structured so that they allow embolic devices and agents to pass through the valve assembly to a location outside the stent or stent system, but prohibit materials from passing outside of the stent, stent graft, or stent system to the interior of the stent, stent graft, or stent system. The thickness Tᵥ of the valve housing 40 may range from about 1 mm to about 20 mm, typically from about 2 mm to about 4 mm. Preferably, the thickness Tᵥ of the valve housing 40 will be about 2 mm. FIG. 9 shows a cross-sectional view of the one-way valve assembly 38 of FIG. 8. FIG. 9 shows the four valve flaps 42 structured and arranged in close proximity to one another in a “pie” formation, however, it is to be understood that the valve flaps 42 may be structured and arranged in many different configurations and any number of valve flaps 42 may be used, all being within the scope of the present invention.

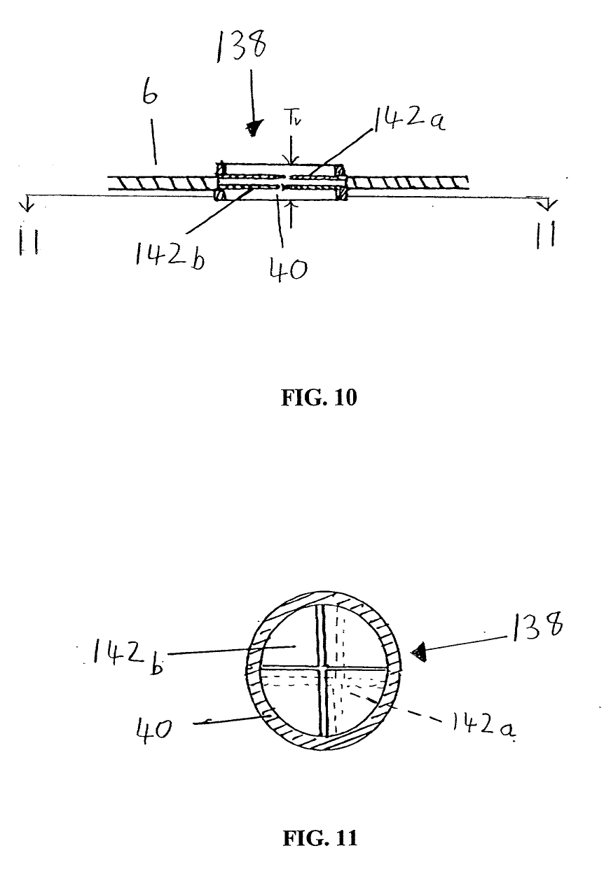

FIG. 10 shows a side sectional view of a portion of a sidewall 6 of a stent 2 containing a one-way valve assembly 138 in accordance with another embodiment of the present invention. As shown in FIG. 10, a first and second layer of valve flaps 142a and 142b may be arranged within the valve housing 40. The two layers of valve flaps 142a and 142b may be placed off center with respect to each other, so that the small spaces between the valve flaps 142a of the first layer do not line up with the small spaces between the valve flaps 142b of the second layer. FIG. 11 is a cross-sectional view of FIG. 10 and shows the valve flaps 142a and 142b arranged within the valve housing 40 of the valve assembly 138. The second layer of valve flaps 142b is shown in solid lines, and the first layer of valve flaps 142a is shown in broken lines situated behind the first layer and slightly off center from the first layer. In this embodiment, the double layer of valve flaps 142a and 142b helps to ensure the one-way operation of the valve assembly 138.

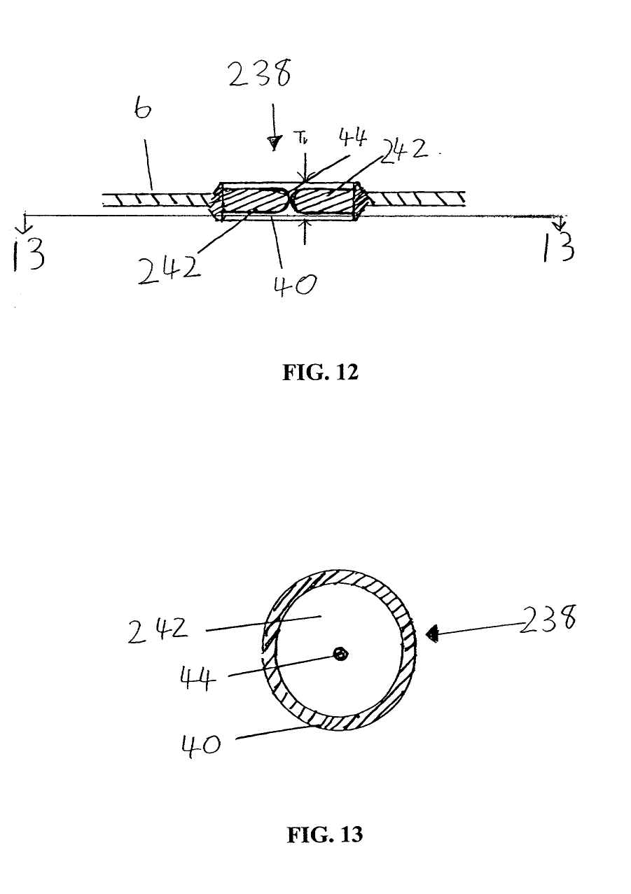

FIGS. 12 and 13 show a one-way valve assembly 238 extending through a portion of a sidewall 6 in accordance with another embodiment of the present invention. In this embodiment, the valve element 242 is a single continuous piece of material with a valve hole 44 located at the center of the valve element 242. As shown most clearly in FIG. 13, the valve hole 44 may be located at the center of the valve element 242 and will allow a delivery device (not shown) to extend through the one-way valve assembly 238.

The valve elements 42, 142, and 242 illustrated in FIGS. 8-13 may be made out of any suitable material, such as but not limited to Polyethylene compounds, Polyurethane, polyethylene terephthalate (PET) and/or polytetrafluorethylene (PTFE), Dacron, polypropylene, silicone, prosthetic material, cadaveric material, umbilical material, and/or various metalic compounds.

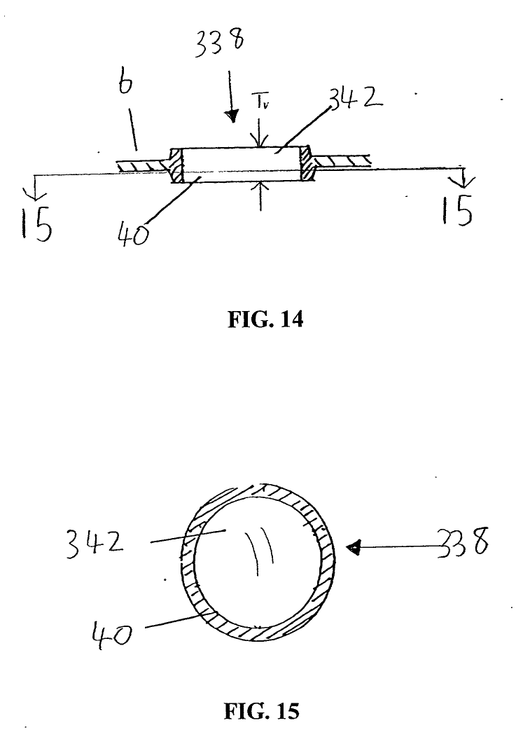

FIGS. 14 and 15 show a one-way valve assembly 338 in accordance with another embodiment of the present invention. FIG. 14 is a side sectional view of a one-way valve assembly 338 extending through a portion of the sidewall 6. The one-way valve assembly 338 includes a valve housing 40 and a valve membrane 342. In this embodiment, the valve membrane 342 may be a solid piece of self-sealing material, such as a Polyethylene compound, Polyurethane, polyethylene terephthalate (PET) and/or polytetrafluorethylene (PTFE), Dacron, polypropylene, silicone, prosthetic material, cadaveric material, umbilical material, and/or various metalic compounds, however any suitable self-sealing material can be used and is within the scope of the present invention. The self-sealing valve membrane 342 will allow a sharp guide wire (not shown) or a catheter with a sharp end (not shown) to pierce the valve membrane 342 in order to deliver embolic devices outside of the stent, stent graft or stent system. When the guide wire, catheter, or other appropriate delivery device is removed from the valve membrane 342 of the one-way valve assembly 338, the hole created in the valve membrane 342 will re-seal. FIG. 15 is a cross-sectional view of FIG. 14 showing the valve housing 40 and the self-sealing valve membrane 342 of the one-way valve assembly 338.

Although a stent 2 is shown in FIGS. 8-15, it is to be understood that the stent could be replaced with the endovascular stent graft 12. Alternatively, the endovascular stent system 28 could be provided instead of the stent 2 alone.

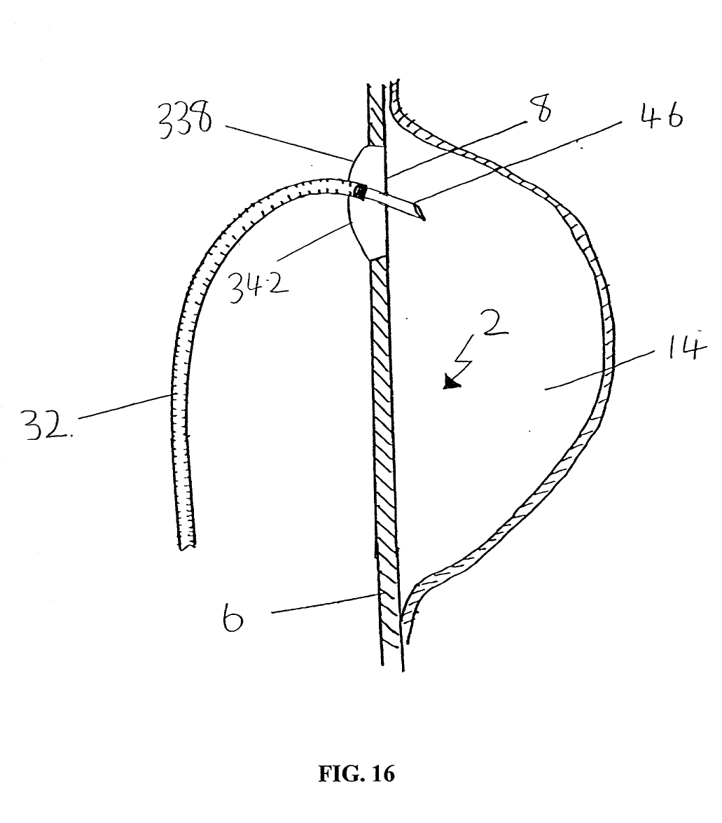

FIG. 16 is a side sectional view of a portion of a sidewall 6 of an endovascular stent 2 containing a port 8 having a self-sealing one-way valve assembly 338 as shown in FIGS. 14 and 15. Although a stent 2 is shown in FIG. 16, it is to be understood that the stent could be replaced with the endovascular stent graft 12. Alternatively, the endovascular stent system 28 could be provided instead of the stent 2 alone. FIG. 16 shows a sharp guide wire 46 piercing the self-sealing valve membrane 342 of the one-way valve assembly 338 and extending through the sidewall 6 and into the space occupied by an aneurysm 14. FIG. 16 also shows a delivery device 32, such as a catheter, being advanced over the sharp guide wire 46.

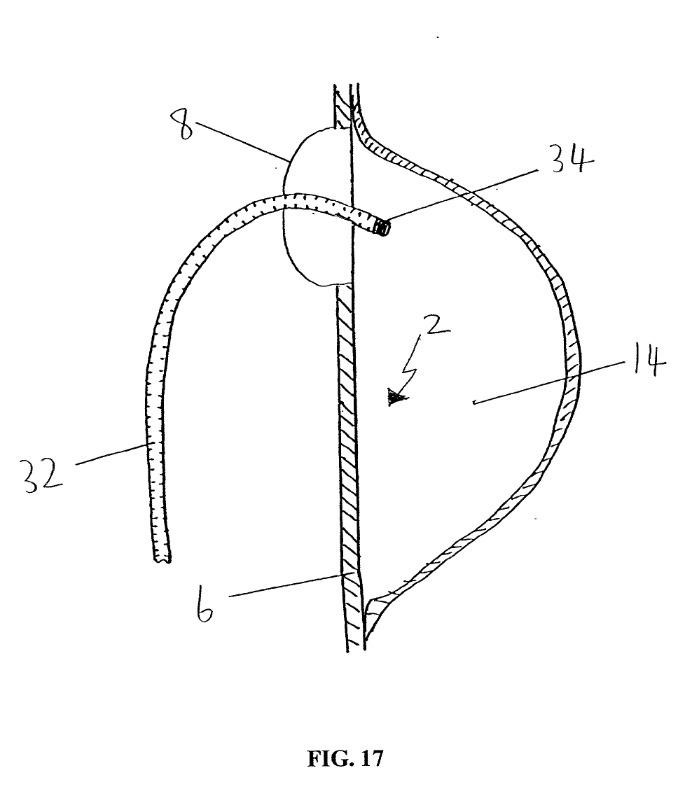

FIG. 17 shows that the delivery device 32 illustrated in FIG. 16 has been advanced far enough along the sharp guide wire (not shown) such that the delivery device tip 34 has been extended through the side wall 6 and into the space occupied by the aneurysm 14. In FIG. 17, the sharp guide wire 46 has been removed, and the delivery device 32 is now ready to deliver embolic devices or agents into the space occupied by the aneurysm 14.

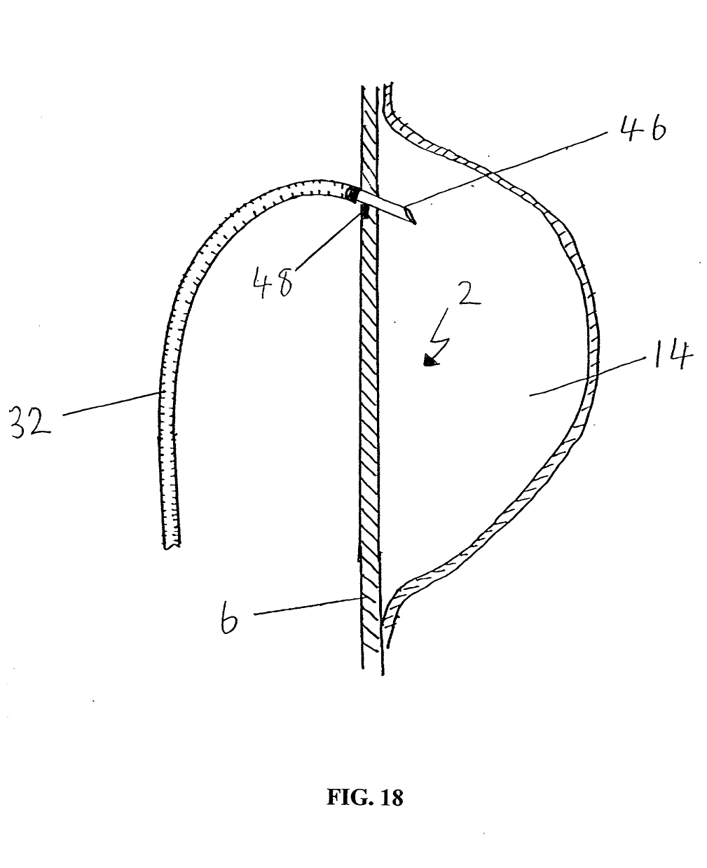

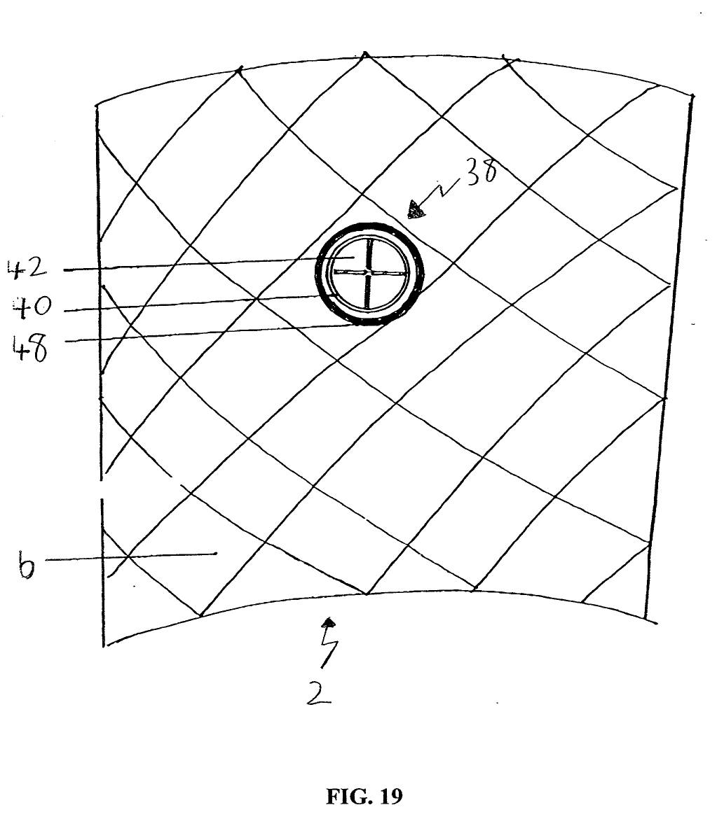

FIG. 18 is a side sectional view of a portion of a sidewall 6 of an endovascular stent 2 in accordance with another embodiment of the present invention, however, an endovascular stent graft 12 or stent system 28 may also be used. In this embodiment, the entire sidewall 6 may be made of a self sealing material, effectively turning the entire sidewall 6 into a port. As shown in FIG. 18, a sharp guide wire 46 may pierce the self-sealing sidewall 6 at any desired location, and a delivery device 32 may then be advanced over the sharp guide wire 46. A radiopaque marker 48 may be placed on portions of the sidewall 6 where it would be desirable to insert the sharp guide wire 46.

FIG. 19 illustrates a sidewall 6 of an endovascular stent 2. An endovascular stent graft 12 and/or stent system 28 may alternatively be provided. The sidewall 6 includes a one-way valve assembly 38 as shown in FIGS. 8 and 9. The one-way valve assembly 38 includes a valve housing 40 and valve flaps 42. The one-way valve assembly 38 may also optionally include a radiopaque marker 48 to aid in locating the one-way valve assembly 38.

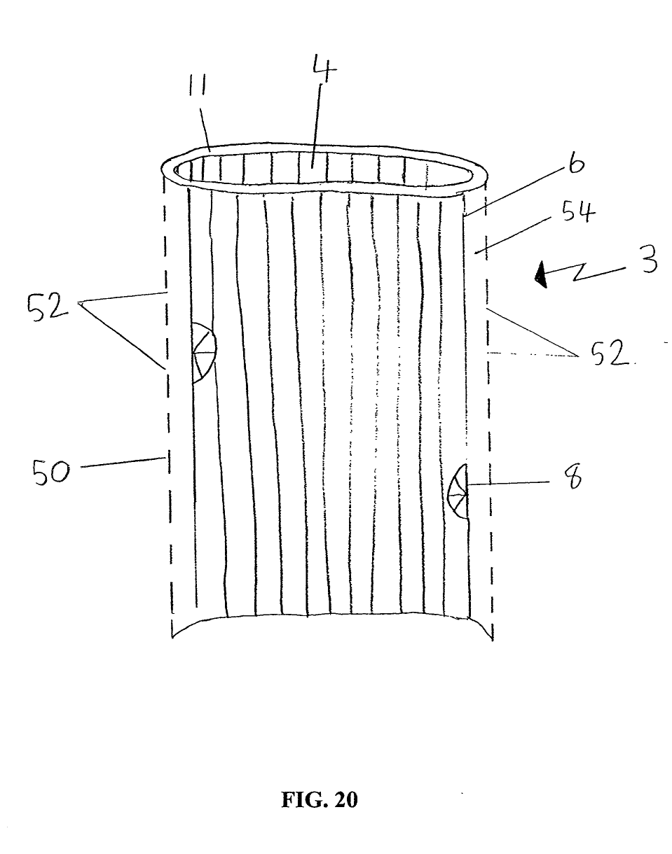

FIG. 20 schematically illustrates an endovascular stent 3 in accordance with another embodiment of the present invention. The endovascular stent 3 includes a first lumen 4 having a perimeter and sidewalls 6 extending around substantially the entire perimeter and at least one port 8 extending through the sidewalls 6 of the first lumen 4 to allow embolic devices to be delivered outside the endovascular stent 3, similar to the embodiment shown in FIG. 1. The endovascular stent 3 also includes an annular ring 11 at the cephalic end 58 of the stent 3, and an outer covering 50, which substantially surrounds the endovascular stent 3 and is attached to the annular ring 11 as shown in FIG. 20. The outer covering may be made of Dacron, woven-polyester, silicone, polyurethane, polyethylene, or the like. As also shown in FIG. 20, the outer covering 50 contains a plurality of pores 52, and the outer covering 50 is structured and arranged so that a cavity 54 is created between the sidewalls 6 of the first lumen 4 and the outer covering 50.

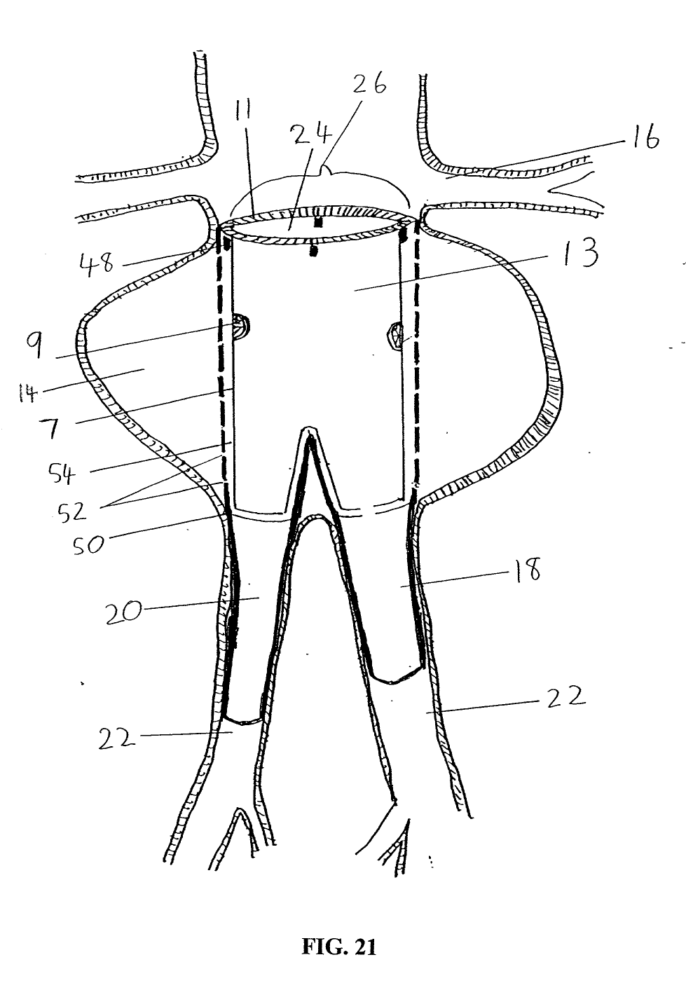

FIG. 21 illustrates an endovascular stent graft 13 in accordance with another embodiment of the present invention. The endovascular stent graft 13 includes a second lumen 24 having a perimeter and sidewalls 7 extending around substantially the entire perimeter, and at least one port 9 extending through the sidewalls 7 of the second lumen 24 to allow for embolic devices to be delivered outside the endovascular stent graft 13. The endovascular stent graft 13 also includes a distal section 26 that is structured to be optionally attached to the caudal section of an endovascular stent, and an ipsilateral section 18 and a contralateral section 20 which are structured to be disposed within iliac arteries 22. As illustrated in FIG. 21, the endovascular stent graft 13 also includes an annular ring 11 at the end of the distal section 26, and outer covering 50, similar to that shown in FIG. 20, that substantially surrounds the endovascular stent graft 13 and is attached to the annular ring 11. The outer covering 50 includes a plurality of pores 52, and the outer covering 50 is structured and arranged so that a cavity 54 is created between the side walls 7 of the second lumen 24 and the outer covering 50.

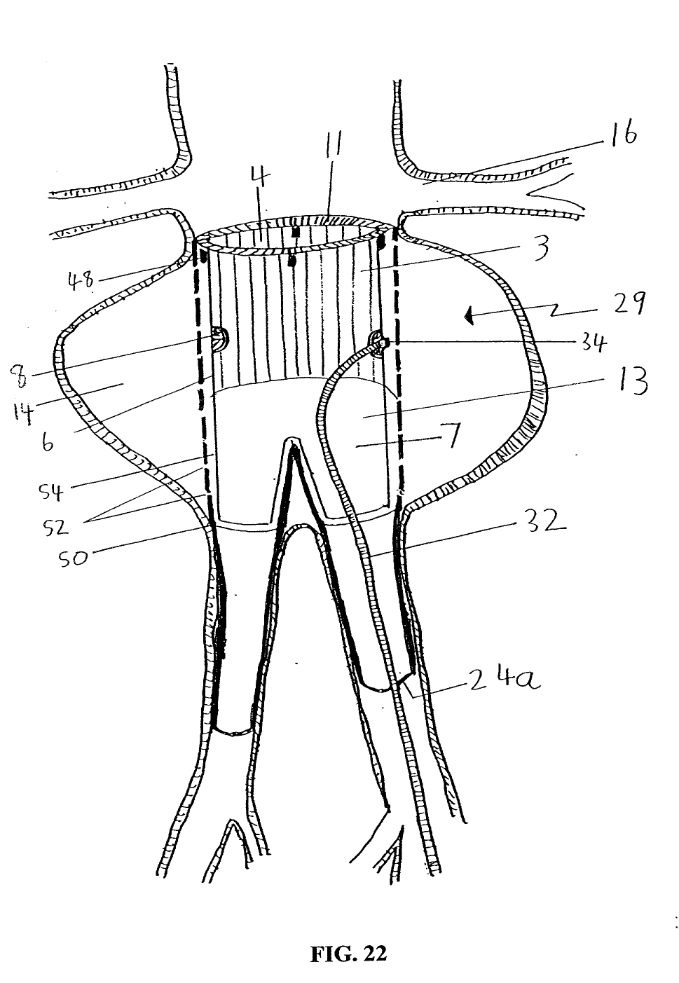

The endovascular stent 3 of FIG. 20 and the endovascular stent graft 13 of FIG. 21 may be combined to form the stent system 29 shown in FIG. 22. As shown in FIG. 22, when the endovascular stent 3 and the endovascular stent graft 13 are attached, the resulting system 29 will be substantially surrounded by an outer covering 50. As shown in FIG. 22, the outer covering 50 contains a plurality of pores 52, and the outer covering is structured and arranged to create a cavity 54 between the sidewalls 6 of the first lumen 4, the sidewalls 7 of the second lumen (not shown), and the outer covering 50. As also illustrated in FIG. 22, a delivery device 32 may be inserted into the port 8 so that the tip 34 of the delivery device 32 extends through the port 8 and the sidewalls 6 into the cavity 54 between the sidewalls 6 and 7 and the outer covering 50. FIG. 22 also shows that optional radiopaque markers 48 may be placed at various locations on the stent system 29.

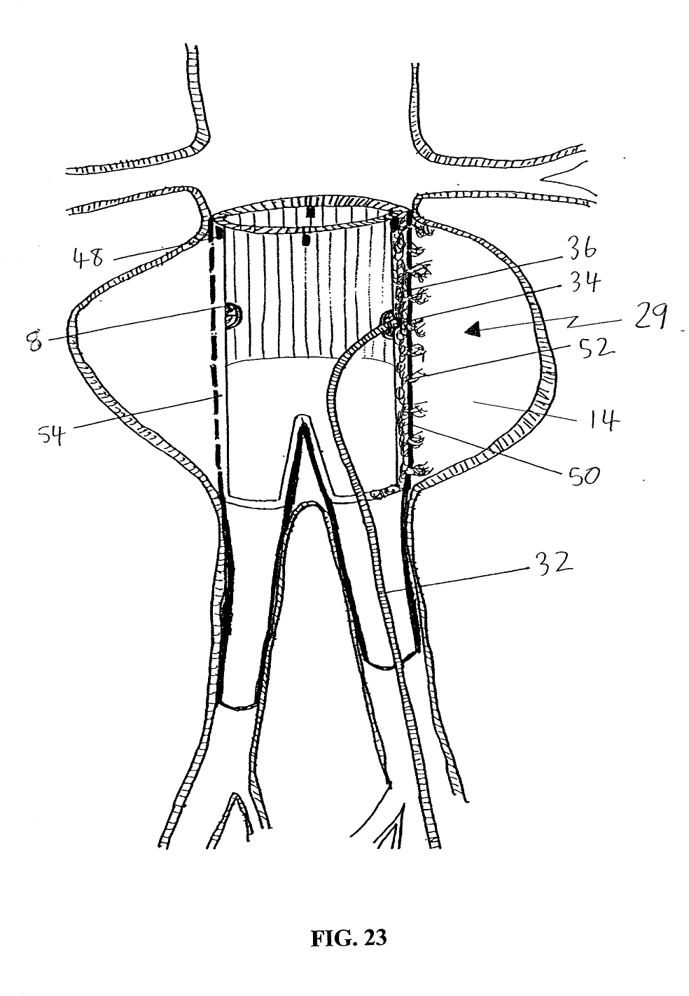

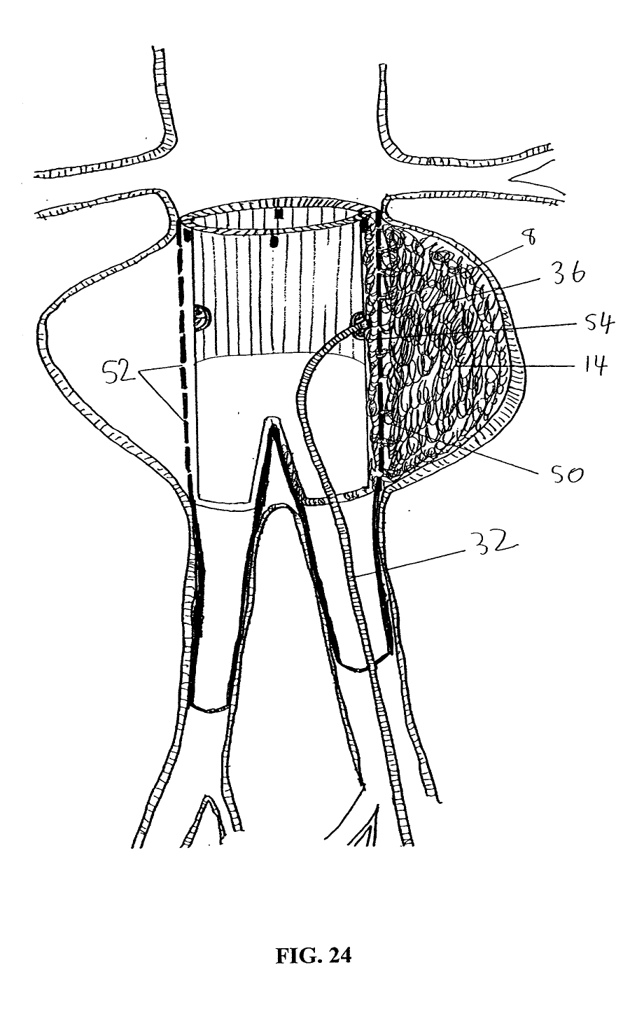

FIGS. 23 and 24 show the stent system 29 of FIG. 22 being used to treat an abdominal aortic aneurysm 14, in accordance with an embodiment of the invention.

FIG. 23 shows an embolic agent 36 being delivered into the cavity 54 by the delivery device 32. As the cavity 54 becomes substantially filled with embolic agent 36, the embolic agent 36 begins to migrate through the pores 52 of the outer covering 50 and into the space occupied by aneurysm 14. FIG. 24 shows that the embolic agent 36 has substantially filled the cavity 54, has migrated through the pores 52 of the outer covering 50, and has substantially filled the space occupied by aneurysm 14. The benefit of such a stent system with a porous outer covering is that the stent system 29 will truly be one-way, and thus blood and other materials will be prevented from leaking into the endovascular stent and/or the endovascular stent graft. An additional benefit is that there would be a more equal distribution of embolic agent 36 throughout the aneurysm 14.

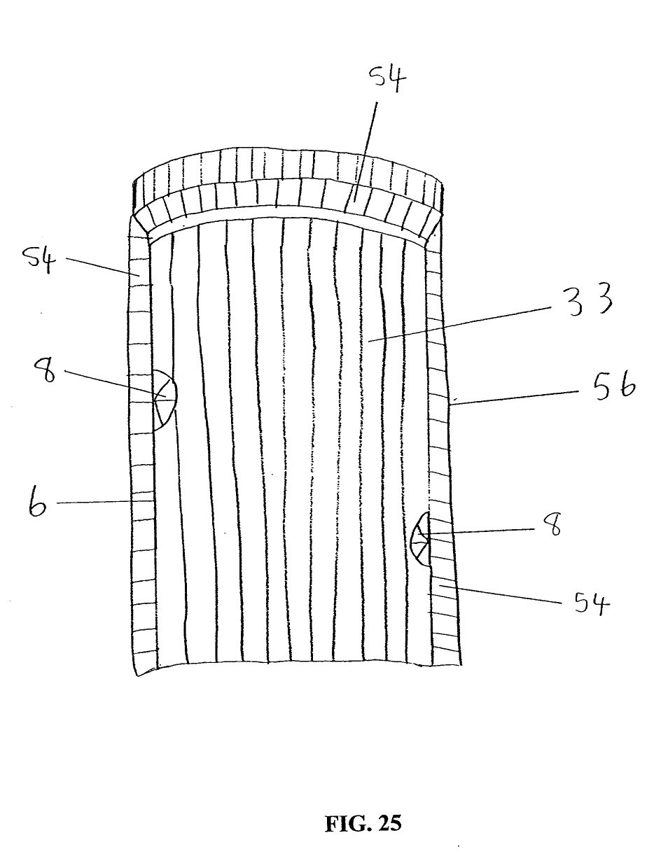

FIG. 25 illustrates an endovascular stent 33 in accordance with another embodiment of the present invention. The endovascular stent 33 includes a first lumen (not shown) having a perimeter and sidewalls 6 extending around substantially the entire perimeter and at least one port 8 extending through the sidewalls 6 of the first lumen to allow embolic devices to be delivered outside the endovascular stent 33. The stent 33 also includes a non-porous outer covering 56, which substantially surrounds the endovascular stent 33 as shown in FIG. 25. The outer covering 56 is structured and arranged so that a cavity 54 is created between the sidewalls 6 of the first lumen and the outer covering 56.

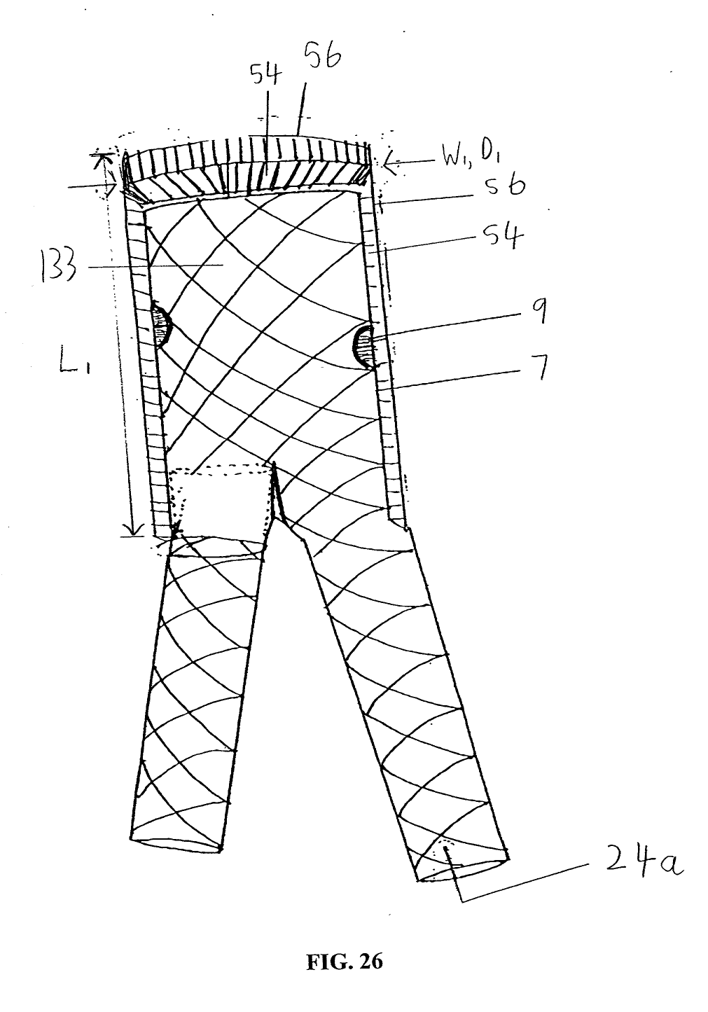

FIG. 26 illustrates an endovascular stent graft 133 in accordance with another embodiment of the present invention. The endovascular stent graft 133 includes a second lumen (not shown) having a perimeter and sidewalls 7 extending around substantially the entire perimeter, and ports 9 extending through the sidewalls 7 of the second lumen to allow for embolic devices to be delivered outside the endovascular stent graft 133. The endovascular stent graft 133 also includes an ipsilateral section 18 and a contralateral section 20 which are structured to be disposed within iliac arteries 22. As illustrated in FIG. 26, the endovascular stent graft 133 also includes a non-porous outer covering 56 that substantially surrounds the endovascular stent graft 133. The outer covering 56 may be structured and arranged so that a cavity 54 is created between the sidewalls 7 of the second lumen and the outer covering 56. Although not shown, the ipsilateral section 18 and the contralateral section 20 of the stent graft 133 may also be substantially surrounded by the outer covering 56.

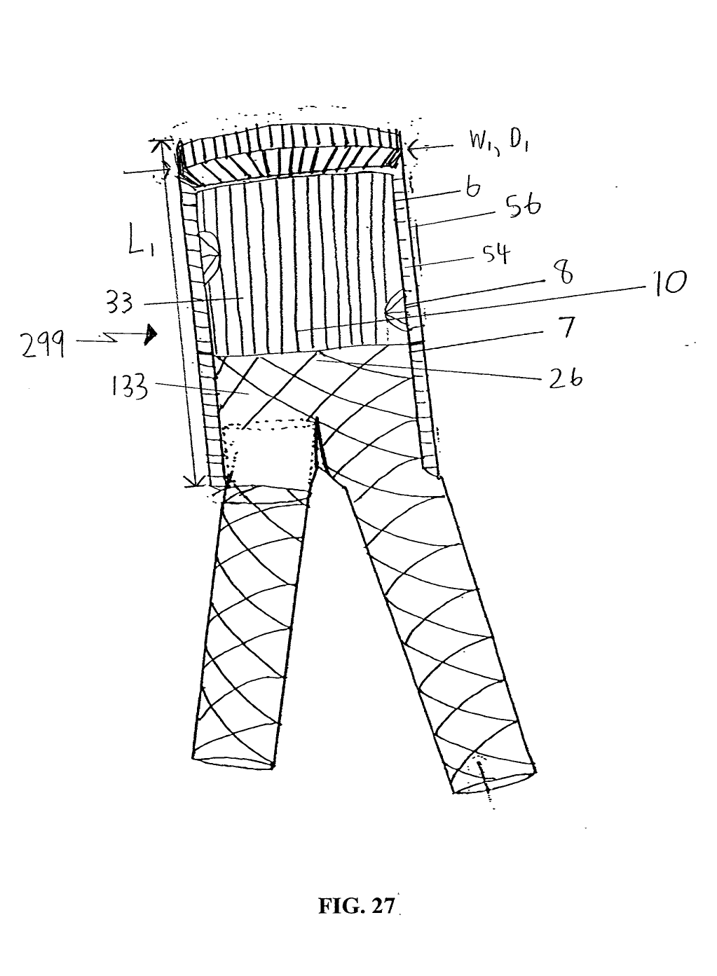

The endovascular stent 33 of FIG. 25 and the endovascular stent graft 133 of FIG. 26 may be combined to form the stent system 299 shown in FIG. 27. In FIG. 27 the distal section 26 of the stent graft 133 is not surrounded by the outer covering 56, and the distal section 26 is structured to be attached to the caudal section 10 of the endovascular stent 33. As shown in FIG. 27, when the endovascular stent 33 and the endovascular stent graft 133 are attached, the resulting system 299 will be substantially surrounded by an outer covering 56. As shown in FIG. 27, the outer covering 56 is structured and arranged to create a cavity 54 between the sidewalls 6 of the first lumen (not shown), the sidewalls 7 of the second lumen (not shown), and the outer covering 56. A delivery device (not shown) may be inserted into the port 8 so that the tip of the delivery device (not shown) extends through the port 8 and the sidewalls 6 into the cavity 54 between the sidewalls 6 and the outer covering 56.

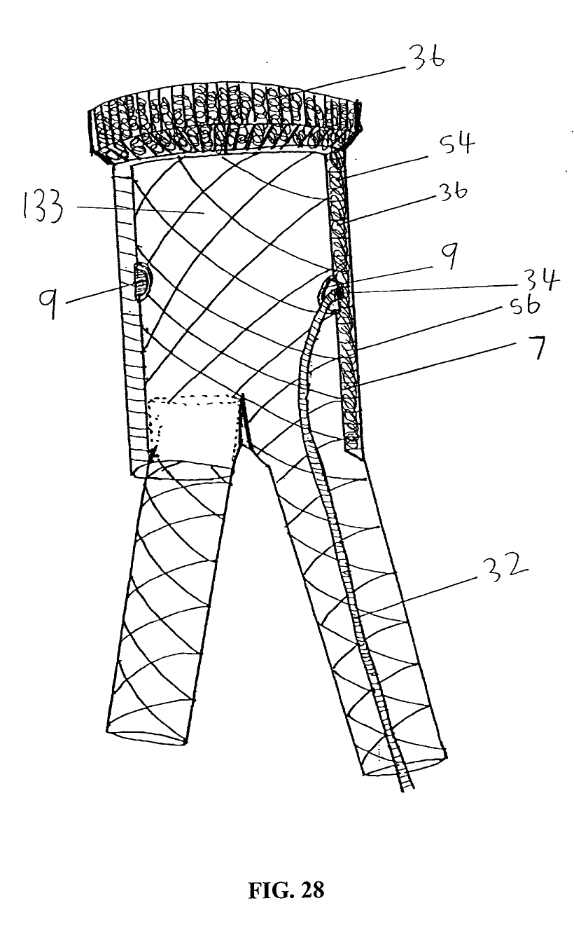

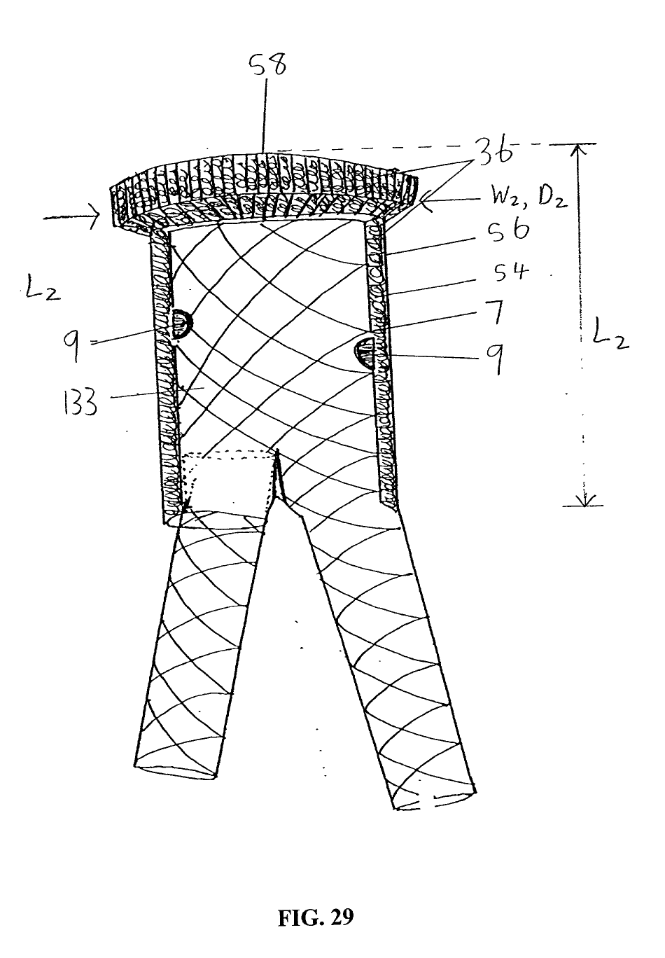

FIGS. 28 and 29 show an endovascular stent graft 133 being used to treat an abdominal aortic aneurysm (not shown), in accordance with an embodiment of the present invention.

FIG. 28 shows an embolic agent 36 being delivered into the cavity 54 by the delivery device 32. As the cavity 54 becomes substantially filled with embolic agent 36, the non-porous outer covering 56 will begin to expand in length, width, and diameter. As the non-porous outer covering begins to expand in substantially all directions, the endovascular stent graft 133 will become firmly seated within the blood vessel, and an aneurysm (not shown) will be effectively sealed, reducing the possibility of an endoleak occurring. FIG. 29 shows that the embolic agent 36 has substantially filled the cavity 54, and the outer covering 56 has substantially increased in length, width, and diameter. FIG. 29 also shows that the cephalic end 58 of the endovascular stent graft 133 has increased in size the most.

As shown in FIG. 27, prior to delivering an embolic agent 36 into the cavity 54, the outer covering 56 may range in length L₁ from about 12 cm to about 25 cm, typically from about 13 cm to about 16 cm. Preferably, the length L₁ will be about 14 cm. Prior to delivering an embolic agent 36 into the cavity 54, the largest cross-sectional area of the outer covering 56 may range in width W₁ from about 18 mm to about 35 mm, typically from about 24 mm to about 30 mm. Preferably, the width W₁ will be about 28 mm. Prior to delivering an embolic agent 36 into the cavity 54, the largest cross-sectional area of the outer covering 56 may range in diameter D₁ from about 18 mm to about 35 mm, typically from about 24 mm to about 30 mm. Preferably, the diameter D₁ will be about 28 mm.

As shown in FIG. 29, after an embolic agent 36 has substantially filled the cavity 54, the outer covering 56 may range in length L₂ from about 12 cm to about 28 cm, typically from about 14 cm to about 17 cm. Preferably, the length L₂ will be about 14 cm. After an embolic agent 36 has substantially filled the cavity 54, the largest cross-sectional area of the outer covering 56 may range in width W₂ from about 19 mm to about 40 mm, typically from about 25 mm to about 30 mm. Preferably, the width W₂ will be about 28 mm. After an embolic agent 36 has substantially filled the cavity 54, the largest cross-sectional area of the outer covering 56 may range in diameter D₂ from about 19 mm to about 40 mm, typically from about 25 mm to about 30 mm. Preferably, the diameter D₂ will be about 28 mm.

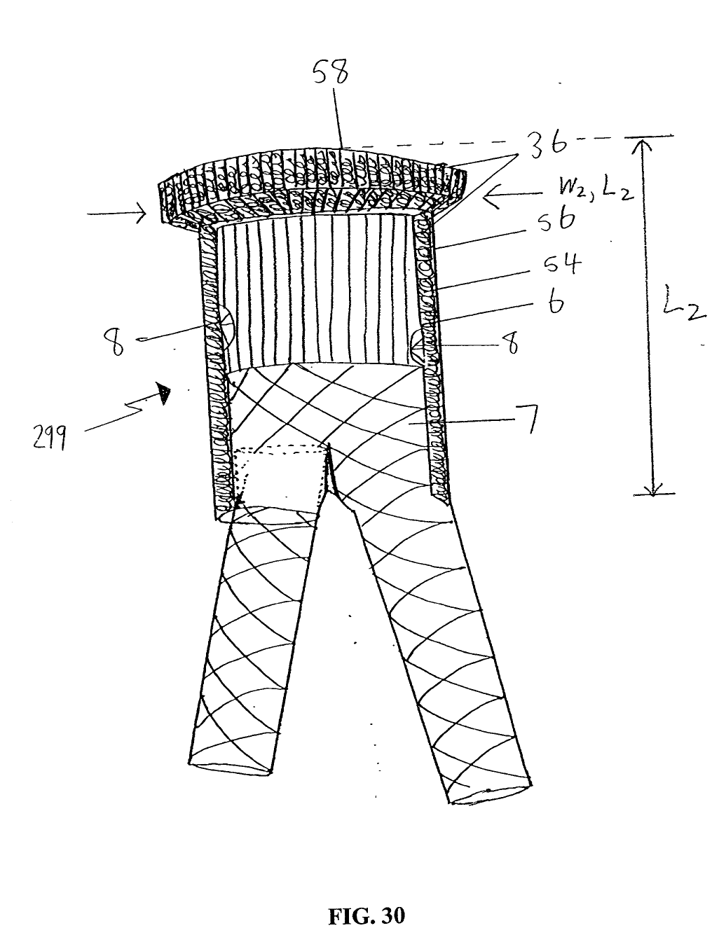

FIG. 30 shows a stent system 299 being used to treat an abdominal aortic aneurysm (not shown), in accordance with an embodiment of the present invention. In FIG. 30, an embolic agent 36 has been delivered into the cavity 54 by a delivery device (not shown). FIG. 30 shows that the embolic agent 36 has substantially filled the cavity 54, and the outer covering 56 has substantially increased in length, width, and diameter. FIG. 30 also shows that the cephalic end 58 of the stent system 299 has increased in size the most.

In one embodiment, the endovascular stent 2, endovascular stent graft 12, and/or stent system 28 of the present invention may be coated with a material to promote proper cell growth. An example of such a material may be, but is not limited to, polytetrafluoroethylene.

Although the delivery device of the present invention has been primarily discussed as delivering embolic agents, such as medical grade glues, it is to be understood that other types of embolic devices may be delivered by the delivery device and are within the scope of the present invention. Examples may include, but are not limited to, suction for aspirating thromboembolic material, and/or diagnostic instruments.

Although the present invention has been primarily described for use in treating abdominal aortic aneurysms and aneurysms of the iliac arteries, it is to be understood that the apparatus and method of the present invention can be used with any endovascular treatment modality or system that would benefit from the advantages disclosed herein and is within the scope of the present invention.

Whereas particular embodiments of this invention have been described above for purposes of illustration, it will be evident to those skilled in the art that numerous variations of the details of the present invention may be made without departing from the invention as defined in the appended claims.

1. An endovascular stent comprising:
a lumen having a sidewall; and
at least one port extending through the sidewall of the lumen structured and arranged to allow for delivery of an embolic device outside the stent.

2. The endovascular stent of claim 1 wherein the port comprises a one-way valve assembly.

3. The endovascular stent of claim 2 wherein the one-way valve assembly has a thickness measured from an outer portion of the sidewall to an inner portion of the sidewall from about 1 mm to about 20 mm.

4. The endovascular stent of claim 1 further comprising a caudal section structured to be attached to a secondary stent graft.

5. The endovascular stent of claim 1 wherein the port includes a radiopaque marker.

6. The endovascular stent of claim 1 wherein the port is structured to receive a delivery device.

7. The endovascular stent of claim 6 wherein the delivery device is a catheter.

8. The endovascular stent of claim 6 wherein the delivery device is a medical grade tube.

9. The endovascular stent of claim 6 wherein the delivery device is a guide wire.

10. The endovascular stent of claim 6 wherein the delivery device delivers embolic devices.

11. The endovascular stent of claim 6 wherein the port comprises a membrane and the delivery device is structured to pierce the membrane.

12. The endovascular stent of claim 6 wherein the delivery device is structured to pierce the sidewalls.

13. The endovascular stent of claim 1 further comprising an outer covering substantially surrounding the stent;
wherein the outer covering is structured and arranged to create a cavity between the sidewalls of the lumen and the outer covering.

14. The endovascular stent of claim 13 wherein the outer covering is porous.

15. The endovascular stent of claim 14 wherein the outer covering is structured to allow the embolic device to extend through the outer covering into a space occupied by an aneurysm.

16. The endovascular stent of claim 13 wherein the outer covering is non-porous.

17. The endovascular stent of claim 13 wherein the outer covering expands longitudinally when an embolic device is delivered outside the stent.

18. The endovascular stent of claim 13 wherein the outer covering expands in diameter when an embolic device is delivered outside the stent.

19. The endovascular stent of claim 13 wherein the outer covering expands in width when an embolic device is delivered outside the stent.

20. An endovascular stent comprising:
a lumen having a sidewall; and
means to allow for the delivery of an embolic device outside the stent and into a space occupied by an aneurysm.

21. The endovascular stent of claim 20 wherein the means for the injection of material outside the stent and into a space occupied by an aneurysm is at least one port extending through the sidewalls of the first lumen.

22. The endovascular stent of claim 20 wherein the means for the injection of material outside the stent and into a space occupied by an aneurysm is at least one one-way valve assembly extending through the sidewalls of the first lumen.

23. An endovascular stent graft comprising:
a lumen comprising a sidewall and having a distal section, and proximate ipsilateral and contralateral sections structured to be disposed within iliac arteries; and
at least one port extending through the sidewall of the lumen structured and arranged to allow for delivery of an embolic device outside the stent graft.

24. The endovascular stent graft of claim 23 wherein the distal section is structured to be attached to a caudal section of a stent.

25. The endovascular stent graft of claim 23 wherein the lumen extends substantially from the distal section to the ipsilateral and contralateral sections.

26. The endovascular stent graft of claim 23 wherein the port comprises a one-way valve assembly.

27. The endovascular stent graft of claim 26 wherein the one-way valve assembly has a thickness measured from an outer portion of the sidewall to an inner portion of the sidewall from about 1 mm to about 20 mm.

28. The endovascular stent graft of claim 23 wherein the port includes a radiopaque marker.

29. The endovascular stent graft of claim 23 wherein the port is structured to receive a delivery device.

30. The endovascular stent graft of claim 29 wherein the delivery device is a catheter.

31. The endovascular stent graft of claim 29 wherein the delivery device is a medical grade tube.

32. The endovascular stent graft of claim 29 wherein the delivery device is a guide wire.

33. The endovascular stent graft of claim 29 wherein the delivery device delivers an embolic device.

34. The endovascular stent graft of claim 29 wherein the port comprises a membrane and the delivery device is structured to pierce the membrane.

35. The endovascular stent graft of claim 29 wherein the delivery device is structured to pierce the sidewalls.

36. The endovascular stent graft of claim 23 further comprising an outer covering substantially surrounding the stent graft;
wherein the outer covering is structured and arranged to create a cavity between the sidewalls of the lumen and the outer covering.

37. The endovascular stent graft of claim 36 wherein the outer covering is porous.

38. The endovascular stent graft of claim 37 wherein the outer covering is structured to allow the embolic device to extend through the outer covering into a space occupied by an aneurysm.

39. The endovascular stent graft of claim 36 wherein the outer covering is non-porous.

40. The endovascular stent graft of claim 39 wherein the outer covering expands longitudinally when an embolic device is delivered outside the stent.

41. The endovascular stent graft of claim 39 wherein the outer covering expands in diameter when an embolic device is delivered outside the stent.

42. The endovascular stent graft of claim 39 wherein the outer covering expands in width when an embolic device is delivered outside the stent.

43. An endovascular stent system comprising:
an endovascular stent comprising:
a lumen having a sidewall; and
at least one port extending through the sidewall of the lumen structured and arranged to allow for delivery of an embolic device outside the stent; and
an endovascular stent graft comprising:
a lumen comprising a sidewall and having a distal section, and proximate ipsilateral and contralateral sections structured to be disposed within iliac arteries; and
at least one port extending through the sidewall of the lumen structured and arranged to allow for delivery of an embolic device outside the stent graft.

44. The endovascular stent system of claim 43 wherein the port of the stent comprises a one-way valve assembly.

45. The endovascular stent system of claim 44 wherein the one-way valve assembly has a thickness measured from an outer portion of the sidewall of the stent to an inner portion of the sidewall of the stent from about 1 mm to about 20 mm.

46. The endovascular stent system of claim 43 wherein the port of the stent includes a radiopaque marker.

47. The endovascular stent system of claim 43 wherein the port of the stent is structured to receive a delivery device.

48. The endovascular stent system of claim 47 wherein the delivery device is a catheter.

49. The endovascular stent system of claim 47 wherein the delivery device is a medical grade tube.

50. The endovascular stent system of claim 47 wherein the delivery device is a guide wire.

51. The endovascular stent system of claim 47 wherein the delivery device delivers embolic devices.

52. The endovascular stent system of claim 47 wherein the port of the stent comprises a membrane and the delivery device is structured to pierce the membrane.

53. The endovascular stent system of claim 47 wherein the delivery device is structured to pierce the sidewalls of the stent.

54. The endovascular stent system of claim 43 wherein the lumen of the stent graft extends substantially from the distal section to the ipsilateral and contralateral sections.

55. The endovascular stent system of claim 43 wherein the port of the stent graft comprises a one-way valve assembly.

56. The endovascular stent system of claim 55 wherein the one-way valve assembly has a thickness measured from an outer portion of the stent graft sidewall to an inner portion of the stent graft sidewall from about 1 mm to about 20 mm.

57. The endovascular stent system of claim 43 wherein the port of the stent graft includes a radiopaque marker.

58. The endovascular stent system of claim 43 wherein the port of the stent graft is structured to receive a delivery device.

59. The endovascular stent system of claim 58 wherein the delivery device is a catheter.

60. The endovascular stent system of claim 58 wherein the delivery device is a medical grade tube.

61. The endovascular stent system of claim 58 wherein the delivery device is a guide wire.

62. The endovascular stent system of claim 58 wherein the delivery device delivers an embolic device.

63. The endovascular stent system of claim 58 wherein the port of the stent graft comprises a membrane and the delivery device is structured to pierce the membrane.

64. The endovascular stent system of claim 58 wherein the delivery device is structured to pierce the sidewalls of the stent graft.

65. The endovascular stent system of claim 43 further comprising an outer covering substantially surrounding the stent system;
wherein the outer covering is structured and arranged to create a cavity between the sidewall of the stent and the sidewall of the stent graft and the outer covering.

66. The endovascular stent system of claim 65 wherein the outer covering is porous.

67. The endovascular stent system of claim 66 wherein the outer covering is structured to allow the embolic device to extend through the outer covering into a space occupied by an aneurysm.

68. The endovascular stent system of claim 65 wherein the outer covering is non-porous.

69. The endovascular stent system of claim 68 wherein the outer covering expands longitudinally when an embolic device is delivered outside the stent system.

70. The endovascular stent system of claim 68 wherein the outer covering expands in diameter when an embolic device is delivered outside the stent system.

71. The endovascular stent system of claim 68 wherein the outer covering expands in width when an embolic device is delivered outside the stent system.

72. A method of using an endovascular stent for embolizing an aneurysm comprising the steps of:
providing an endovascular stent having a lumen comprising a sidewall with at least one port extending therethrough;
positioning the endovascular stent within an artery adjacent to an aneurysm;
guiding a delivery device into the endovascular stent;
inserting the delivery device into the port so that at least a portion of the delivery device extends through the sidewall of the lumen; and
delivering an embolic device through the delivery device to a location outside the stent.

73. A method according to claim 72 wherein the port comprises a one-way valve assembly.

74. A method according to claim 72 wherein the embolic device is delivered into a space occupied by an aneurysm.

75. A method according to claim 72 wherein the stent further comprises a caudal section structured to be attached to a secondary stent graft.

76. A method of using an endovascular stent graft for embolizing an aneurysm comprising the steps of:
providing an endovascular stent graft including a lumen comprising a sidewall and having a distal section, and proximate ipsilateral and contralateral sections, wherein the sidewall includes at least one port extending therethrough;
positioning the endovascular stent graft within an artery adjacent to an aneurysm;
guiding a delivery device into the endovascular stent graft;
inserting the delivery device into the port so that at least a portion of the delivery device extends through the sidewall of the lumen; and
delivering an embolic device through the delivery device to a location outside the stent graft.

77. A method according to claim 76 wherein the endovascular stent graft is modular and is structured to be attached to a caudal section of a stent.

78. A method according to claim 76 wherein the lumen extends substantially from the distal section to the ipsilateral and contralateral sections of the endovascular stent graft.

79. A method according to claim 76 wherein the port comprises a one-way valve assembly.

80. A method according to claim 76 wherein the embolic device is delivered into a space occupied by an aneurysm.

81. A method of using an endovascular stent system for embolizing an aneurysm comprising the steps of:
providing an endovascular stent system comprising:
an endovascular stent having a lumen comprising a sidewall with at least one port extending therethrough; and
an endovascular stent graft including a lumen comprising a sidewall and having a distal section, and proximate ipsilateral and contralateral sections, wherein the sidewall includes at least one port extending therethrough;
positioning the endovascular stent system within an artery adjacent to an aneurysm;
guiding a delivery device into the endovascular stent system;
inserting the delivery device into the port of the stent or the port of the stent graft so that at least a portion of the delivery device extends through the sidewall of the stent or the sidewall of the stent graft; and
delivering an embolic device through the delivery device to a location outside the stent system.

82. A method according to claim 81 wherein the lumen of the endovascular stent graft extends substantially from the distal section to the ipsilateral and contralateral sections of the endovascular stent graft.

83. A method according to claim 81 wherein the port of the stent comprises a one-way valve assembly.

84. A method according to claim 81 wherein the port of the stent graft comprises a one-way valve assembly.

85. A method according to claim 81 wherein the embolic device is delivered into a space occupied by an aneurysm.
